(19) United States
(12) Patent Application Publication
(10) Pub. No.: US 2005/0256187 A1
(43) Pub. Date: Nov. 17, 2005

(54) Method and composition for synergistic topical therapy for neuromuscular pains

(75) Inventor: Rainer K. Liedtke, Gruenwald (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: BIONICS PHARMA GmbH, Gruenwald (DE)

(21) Appl. No.: 11/028,238

(22) Filed: Jan. 4, 2005

(30) Foreign Application Priority Data
Jan. 5, 2004 (DE) 10 2004 1 093.5

(51) Int. Cl. A61K 31/21

(52) U.S. Cl. 514/506

(57) ABSTRACT

The invention relates to a method and composition for synergistic topical therapy of the symptoms of neuromuscular pains. In this method, for intact skin or open skin, there is used a suitable topical pharmaceutical formulation, which is loaded in a suitable dose relationship with a sodium channel blocker from the class of local anesthetics of the ester or amide type and a substance from the class of non-steroidal anti-inflammatory drugs, and which releases these substances selectively onto or under the skin region. By the simultaneous inhibition of the initial inflammatory pain factors at the cellular level and also of the transmission of neuronal pain impulses in reaction thereto, this therapy achieves pharmacologically more effective alleviation of neuromuscular pain.

### BACKGROUND OF THE INVENTION

1. Field of the Invention

The invention relates to a method and composition for synergistic topical therapy of the symptoms of neuromuscular pains close to or far from the joints, especially to a topical pharmaceutical formulation for application on the skin or dermal layers.

2. Description of the Related Art

Neuromuscular pains are emotionally negative perceptions that exist during local traumas in the tissue of the locomotor and musculoskeletal systems or during functional disorders thereof, and that can be caused by physical, chemical, biological or psychosomatic influences. Thus they affect tissue in the form in particular of the functionally involved nerves, muscles, tendons and joints as well as the skin, bone and cartilaginous parts of the skeleton associated therewith via tissue or function. Such syndromes are also known as myalgias or musculoskeletal pains, among other names. In the further context, as well as in the context of such syndromes, however, pains of the aforesaid type will be consistently meant and understood here as neuromuscular pains, used in the functionally generic sense.

It is known that pain symptoms such as occur even in simple muscle strains and tears, in back pains, blunt-force traumas, joint and fascial complaints and even surgical procedures are caused primarily by a local cascade of biochemical reactions in tissue, ultimately culminating in the irritation and functional disorder of peripheral sensory (afferent) nerves.

The prevalence and incidence of painful disorders of neuromuscular type are very high, because of the frequent everyday causes of such disorders in the general population. These disorders, which in many cases also restrict motion, therefore belong to the most frequent causes that lead to reduced fitness or even incapacity for work.

Pharmacologically, these pain symptoms are conventionally predominantly treated with systemically acting oral or injectable antibiotics and anti-inflammatories. The main pharmacological principle for such pain treatment or for restoration of motion functions is currently the use of substances known internationally in medical and pharmaceutical circles as non-steroidal anti-inflammatory drugs or NSAIDs. Substances such as acetylsalicylic acid, diclofenac and ibuprofen belong chemically to the group of NSAIDs. NSAIDs are produced in numerous forms for pharmaceutical application , such as tablets, suppositories or injections for systemic use and also in topical preparations for application on the skin.

In this connection, it is appropriate to begin here by clarifying the term “topical application” as a type of administration that contrasts with systemic application of drugs. Hereinafter, topical application will be used to mean local administration of drugs. In the field of pain therapy, therefore, it comprises administration directly on or in a painful region. This can be the case, for example, of a salve applied over a painful spot on the skin. Thus the point of action is largely identical to the point of administration. This contrasts with systemic administration, in which a drug, taken in the form of a tablet, for example, is distributed via the bloodstream throughout the entire body only after absorption via the stomach and intestines, so that thereafter only a fraction even reaches the point of origin of the pain. Accordingly, the point of administration and the point of action are not identical in systemic administration. The terms used here, such as “topical application form” or “topical composition”, therefore mean pharmaceutical formulations that are prepared for the purpose of topical application.

In systemic therapies using NSAIDs, the rate of adverse effects is generally significant. These effects include in particular gastric disorders and ulcers as well as kidney-function and cardiovascular disorders. Systemic application of NSAIDs for neuromuscular pains is therefore limited by their spectrum of adverse effects.

In topical dermal (transcutaneous) applications of NSAIDs, however, the therapeutic benefit is disputed, even though their rate of secondary effects is much lower, since their analgesic effect is often much smaller. According to prevailing opinion, the decreased efficacy of NSAIDs in topical administration is often blamed on the slower rate of absorption through the skin, and so is interpreted exclusively quantitatively as a dose-effect problem. However, a pharmacodynamic cause must also be suggested here as an alternative to such an assumption. For example, the NSAIDs, in their capacity as anti-inflammatories, cover only certain sections within the overall cascade of the cellular pain mechanism, or in other words those sections which also have an inherent painkilling effect. They include in particular those associated with biochemical inflammatory processes in tissues. For example, the primary effect of NSAIDs is merely to inhibit cyclooxygenases 1 and 2 (COX-1/2), thus reducing prostaglandin formation. Consequently, they are also known as COX inhibitors.

Prostaglandins are hormone-like substances that occur in chemically diverse forms throughout the human body. Because of their very broad spectrum of biological effects, they are integrally involved in the development of inflammatory processes and fever. In connection with inflammations and other phenomena, for example, they increase the sensitivity of pain receptors, influence the contraction of smooth muscles and intensify the release of various hormones.

In contrast, however, the NSAIDs have no direct effects on the subsequent processes of neuronal conduction of pain information from the location at which the peripheral pain originates to the central nervous system, even though such processes are very important for the perception of pain.

This lack of efficacy of the NSAIDs may also be explained by the occurrence of a rate of smaller therapy effects or by non-responders in topical application of NSAIDs. This is the case in particular when the inflammatory component is only slightly pronounced in the tissue.

In pain therapy, local injections with local anesthetics have also proven clinically successful in some areas of application. The basic concept of this approach is to disable nerve pathways by numbing them. In this way, undesired reflex reactions are then supposedly suppressed. However, injection techniques are invasive and also painful in their own right and, because of anatomic considerations, require experienced medical treatment.

A proven pharmacological principle that is further distinguished from the foregoing comprises non-invasive application of local anesthetics with topical patch systems for neuromuscular pains. This new therapy was first discovered by the present inventors and also only introduced in recent years. This was achieved with U.S. Pat. No. 5,776,952 for the field of topical application of local anesthetics for back pains and with U.S. Pat. No. 5,840,755 for topical treatment of headaches. Only in this connection was it also found that local anesthetics not only can be used invasively as conventional numbing agents but also that they can be regarded and used non-invasively as analgesics with specific neural action to combat neuromuscular pains.

The pharmacological mechanism of action of the substances used for this purpose, predominantly local anesthetics of the amide and ester type, of which lidocaine is an example of the amide type, is inhibition of the fast sodium ion influx in nerve fibers; in other words, they act as sodium channel blockers. Through this specific effect they block impulse conduction in nerve fibers, in principle affecting all regional nerve fibers at the same time. However, the fact that the sensory pain-conducting fibers are anatomically thinner than the motor fibers (for example: Strichartz, G. R. (Ed.): Local Anesthetics, Handbook of Experimental Pharmacology, Vol. 81, Springer, Berlin, N.Y. 1987) simultaneously makes it possible to differentiate different active effects on the basis of the administered topical dose.

An additional and important basic aspect for such a dose-saving transcutaneous therapeutic formulation was to regard afferent pain signals as bioelectrically irregular neuronal information samples of the central nervous system that can be regularized or normalized once again, merely by small doses of local anesthetics. It is only in recent years that these predictions and implicit assumptions of U.S. Pat. No. 5,776,952, U.S. Pat. No. 5,840,755 and other publications concerning how ectopic pulses are influenced by lidocaine have been further verified experimentally (Khodorova A., Meissner K., Leeson S., Strichartz G. R.: Muscle Nerve, 24; 634 (2001), Persaud N., Strichartz G. R.: Pain, 99, 333 (2002)).

Alleviation of peripheral pain by topical transcutaneous therapy therefore requires smaller doses of local anesthetics than doses with which the nerve function would be anesthetized, or in other words completely inactivated. Thus transcutaneous analgesia is also distinguished by its selectivity for pain receptors from transcutaneous anesthesia, which influences all local receptors, and thus also brings about inactivation of motor functions, which is undesired in the case of the therapeutic goal of pure analgesia.

For their part, however, these effects of sodium channel blockers of the local-anesthetic type extend specifically only to this section of the neuronal level. In contrast to the NSAIDs, they do not have any pharmacological effects on biochemical inflammatory processes in tissues, nor do they have antipyretic effects.

This absence of effect may therefore explain even the occurrence of a rate of smaller therapeutic effects or even therapeutic non-responders in the topical application of sodium channel blockers of the local-anesthetic type, especially when stronger inflammatory tissue components are involved here.

On the whole therefore, it is evident that the NSAIDs on the one hand and the sodium channel blockers of the local-anesthetic type on the other hand cover only sections—which are very different for each—of the peripheral local pain cascade through the cellular sequences of their pain-alleviating mechanisms of action:

The NSAIDs exert an effect on initial pain-causing events of inflammatory processes in tissues primarily via COX inhibition, however, the sodium channel blockers of the local-anesthetic type do not exert direct effects, especially on the algesic mediators associated with inflammation or edema. Sodium channel blockers of the local anesthetic type exhibit effects that are relevant for pain alleviation only at the level of the neuronal processes of pain transmission and central pain perception that develop after the initial pain causing events such as inflammation and edema.

### BRIEF SUMMARY OF THE INVENTION

The present inventors diligently and comprehensively investigated the cellular sequences occurring in pain and inflammation and inferred that joint administration of an NSAID and of a sodium channel blocker from the class of local anesthetics provides a functional pharmacological synergism perceptible on the whole, despite the conventional understanding that these classes of drugs each have very different mechanisms and act on different cell structures. Therapeutically, joint local or topical administration of these substances provides mutual reinforcement or potentiation of their effects on pain . For example, when a NSAID and sodium channel blocker of the local anesthetic class are topically administered in combination, they synergistically reduce pain levels without completely inactivating nerve function.

### DETAILED DESCRIPTION OF THE INVENTION

One object of the invention is to improve the symptoms of neuromuscular pains close to or far from joints with a synergistic topical therapy on the skin or open dermal layers.

This object is achieved by the fact that, for intact or open dermal layers, there is used a suitable topical pharmaceutical formulation, which contains a therapeutically appropriate dose of a sodium channel blocker from the class of local anesthetics of the ester or amide type and an analgesic from the class of non-steroidal anti-inflammatory drugs, in which the doses of these substances are present in a suitable dose relationship, and which releases the contained substances selectively onto or into the skin region located under the pharmaceutical formulation.

In order to broaden the application of the therapy, the topical pharmaceutical formulation in a further embodiment of the invention is a salve, gel, powder, emulsion, suspension or lotion, an aqueous or alcoholic solution, a spray or a transcutaneous patch system, wherein the active ingredients used in the pharmaceutical formulation can be present homogeneously together or disposed in different chemical phases or in different physical geometries. A suitable topical preparation may be selected from those known in the art. Such topical preparations and methods for their administration are also incorporated by reference to Topical Drug Delivery Formulations, by Osbourne and Amann, Marcel Dekker (1990) or Electrically Assisted Transdermal and Topical Drug Delivery by Ajay K. Banga,, CRC Press (1998).

In order to specify a suitable therapy, the topical pharmaceutical formulation is formulated using active ingredients in the form of lidocaine as the sodium channel blocker from the class of local anesthetics of the amide type and diclofenac as the substance from the class of non-steroidal anti-inflammatory drugs, wherein each of these substances can be present in a concentration of 0.5% to 40%. This range includes all intermediate values and subranges, such as 0.5%, 0.6%, 1%, 2%, 5%, 10%, 15%, 20%, 25%, 30%, 35%, 39% and 40%.

In order to improve the efficacy and tolerance of the therapy, the topical pharmaceutical formulation is formulated using tetracaine, prilocaine, bupivacaine, mepivacaine, etidocaine, procaine, benzocaine, propoxycaine, hydroxyprocaine, chloroprocaine, ambucaine, metabutoxycaine, proparacaine, paraethoxycaine, butacaine, isobucaine, hexylcaine, piridocaine, piperocaine, cyclomethycaine, procainamide, dibucaine, pyrrocaine or tolycaine as further sodium channel blockers from the class of local anesthetics of the amide or ester type, wherein the substance can be present in a concentration of 0.5% to 40%. This range includes all intermediate values and subranges, such as 0.5%, 0.6%, 1%, 2%, 5%, 10%, 15%, 20%, 25%, 30%, 35%, 39% and 40%.

In order to improve the efficacy and tolerance of the topical therapy, the topical pharmaceutical formulation is formulated using indomethacin, piroxicam, ibuprofen, ketoprofen, naproxen, tenoxicam, etofenamate, mefenamine, flufenamic acid, felbinac, salicylic acid, acetylsalicylic acid, methyl salicylate, diethylamine salicylate or hydroxyethyl salicylate as further substances from the class of non-steroidal anti-inflammatory drugs, wherein this substance can be present in a concentration of 0.5% to 40%. This range includes all intermediate values and subranges, such as 0.5%, 0.6%, 1%, 2%, 5%, 10%, 15%, 20%, 25%, 30%, 35%, 39% and 40%.

A composition suitable for topical application may be formulated using a 1:1 ratio of the sodium channel blocker: NSAID or different w/w ratios, ranging for example from 1-80: 80-1. This range includes all intermediate ratios, such as 1:80, 1:40, 1:20, 1:10, 1:5, 1:4, 1:3, 1:2, 1:1, 2:1, 3:1, 4:1, 5:1, 10:1, 20:1, 40:1 or 80:1.

The topical pharmaceutical formulation of the present invention may be used for the treatment of closed neuromuscular pains and joint pains, of muscle bruises, strains and tears, in back pains of neuromuscular origin, rheumatic muscle complaints, myofascial pains and tendonitis, in inflammatory joint irritations, joint distortions and joint compressions.

The topical pharmaceutical formulation may also be used for the treatment of neuromuscular pains based on local nerve irritations or nerve injuries, including the symptoms of neurodermatitis; or for the treatment of neuromuscular pains in open wounds as well as in post-incisional wound pains after surgical procedures.

The topical pharmaceutical formulation may be used medically for humans or for animals in the veterinary area.

Heretofore, a direct synergistic therapy for neuromuscular pains by means of topical pharmaceutical formulations containing both an NSAID (COX inhibitor) and a sodium channel blocker from the class of local anesthetics of the ester or amide type has been neither described nor used for medical purposes. This does not seem surprising, in view of the great differences in the respective pharmacological activity profiles and mechanisms of action of the two substance classes and also their applications in medical practice heretofore.

In addition, non-invasive therapy with local anesthetics in topical patch systems for neuromuscular pains was discovered and introduced by us only in recent years. According to the first therapeutic experiences in this regard, it also became evident that, in the presence of stronger inflammatory components in the tissue, the therapeutic response rate to the sodium channel blockers also seemed to be smaller, and so a pharmacodynamic supplementary effect was needed.

Without a comprehensive analysis of the sequence of cellular events in the development of local pain, including an analysis of inflammatory processes, therefore, the mutually reinforcing benefits of these substance classes for neuromuscular pain therapy is neither perceptible nor obvious. It is only through this analysis that it is found that the new topical therapy generates a synergistic pain alleviation that proceeds by dual mechanisms and that acts both during the phase of pain development and also during its reactive consequence of pain transmission. In this way the substances used then compensate for the respective lack of effect of the other substance class.

In clinical terms, therefore, both the pain-inducing edematous-inflammatory tissue effects and the pain-maintaining irritations that occur during sensory neuronal conduction are regulated. This combination then leads locally to more profound alleviation of the pain symptoms. The breadth of effect of the influence both on the inflammatory and on the neuralgic components therefore cannot be achieved with any of the individual substances alone.

Furthermore, the effect is not possible by systemic administrations of the combination of these substances. The synergistic effect can be adequately developed therapeutically only if the effect of the two components is also achieved by appropriate administration directly at the location of a pain-inducing lesion. Such an effect cannot be adequately controlled in systemic administration. In any case, a high risk of unspecific injury would exist in systemic application. In contrast to the great safety in topical application, systemic application of, for example, a local anesthetic would lead to a high toxic risk, because of its cardiovascular effects among others.

Functionally, the two substances act in the same direction in alleviating pain. However, the fact that they have different points of attack as regards tissue topology suggests the further possible benefit of an additive or superadditive (potentiating) effect, so that the dose can be reduced to achieve an even further improved safety potential.

On the whole, therefore, there is achieved as an advantage of the invention an improved alleviation of the overall symptoms of peripheral neuromuscular pain, since the invention for the first time also permits inhibition of both the tissue-inflammation and neuralgic components of pain, both of which are always present to a certain degree. This discovery was actually made as early as the first individual medical case studies, in which better pain alleviation even in neuromuscular pain syndromes was achieved with topical formulations similar to those of Example 1 hereinafter than was the case with commercial topical formulations containing only individual substances, such as a gel formulation containing 2% diclofenac or a patch containing 5% lidocaine.

### EXAMPLE 1

A general illustrative but non-limitative example of the embodiment of a topical pharmaceutical formulation can be the gel formulation familiar to the person skilled in the pharmaceutical art, which formulation then contains in 100 grams of this gel firstly 2 grams of diclofenac sodium as the NSAID component and secondly 4 grams of lidocaine hydrochloride as the sodium channel blocker component. In this example, therefore, the topical w/w dose relation of NSAID to sodium channel blocker is adjusted to 1:2. Approximately 3 grams of this gel can be used 2 to 4 times daily as needed on the painful intact skin area to be treated.

### EXAMPLE 2

As a further non-limitative example of the embodiment of a topical pharmaceutical formulation, there can be produced a patch formulation familiar to the person skilled in the pharmaceutical art.

The side thereof facing the skin is coated with a polymer that will adhere to the skin. This polymer is used both as a skin adhesive and as a reservoir and control element for release of the constituents. In 1 gram of this adhesive coating there can be contained 25 mg of diclofenac sodium as the NSAID component as well as 50 mg of lidocaine hydrochloride as the sodium channel blocker, each in homogeneous distribution. Because of pharmacokinetic requirements, the concentrations in the topical patch are adjusted to higher levels than in the gel. In this example also, therefore, the w/w dose relation of NSAID to sodium channel blocker is adjusted to 1:2. A patch of this type can be adhered to a painful point of the intact skin for up to 24 hours.

Numerous further pharmaceutical embodiments are possible, for example, by changes of the dose relationships of the two active ingredients in a homogeneous mixture, by pharmaceutical formulations in which the two substances are contained in respectively different phases, or by patches in which the two substances are applied only at geometrically different places.

Modifications and Other Embodiments

Various modifications and variations of the described compositions and their methods of use as well as the concept of the invention will be apparent to those skilled in the art without departing from the scope and spirit of the invention. Although the invention has been described in connection with specific preferred embodiments, it should be understood that the invention as claimed is not intended to be limited to such specific embodiments. Various modifications of the described modes for carrying out the invention which are obvious to those skilled in the pharmaceutical, medical, cosmetic, biological, chemical fields or related fields are intended to be within the scope of the following claims.

### INCORPORATION BY REFERENCE

Each document, patent, patent application or patent publication cited by or referred to in this disclosure is incorporated by reference in its entirety. However, no admission is made that any such reference constitutes prior art and the right to challenge the accuracy and pertinency of the cited documents is reserved. Specifically, German Application No. 10 2004 001 093.5, filed Jan. 5, 2004 is incorporated by reference.

1. A method for synergistic topical therapy for symptom(s) of neuromuscular pain(s) comprising:
administering to a subject in need thereof an effective amount of a composition comprising:
at least one sodium channel blocker(s) from the class of local anesthetics of the ester of amide type, and
one or more analgesic(s) from the class of non-steroidal anti-inflammatory drugs,
wherein said composition is formulated as a topical pharmaceutical composition which releases the drugs onto or into the skin.

2. The method of claim 1 which is a gel, salve, powder, emulsion, suspension or lotion, an aqueous or alcoholic solution, a spray or a transcutaneous patch system.

3. The method of claim 1, wherein the sodium channel blocker(s) and analgesic(s) are present homogeneously together.

4. The method of claim 1, wherein the sodium channel blocker(s) and analgesic(s) are disposed in different chemical phases or in different physical geometries.

5. The method of claim 1, wherein the sodium channel blocker is an amide-type compound.

6. The method of claim 1, wherein the sodium channel blocker is an ester-type compound.

7. The method of claim 1, wherein the sodium channel blocker(s) is selected from the group consisting of tetracaine, prilocaine, bupivacaine, mepivacaine, etidocaine, procaine, benzocaine, propoxycaine, hydroxyprocaine, chloroprocaine, ambucaine, metabutoxycaine, proparacaine, paraethoxycaine, butacaine, isobucaine, hexylcaine, piridocaine, piperocaine, cyclomethycaine, procainamide, dibucaine, pyrrocaine and tolycaine.

8. The method of claim 1, wherein the sodium channel blocker is lidocaine.

9. The method of claim 1, wherein the sodium channel blocker(s) is present in an amount ranging from 0.5% to 40%.

10. The method of claim 1, wherein the analgesic(s) is selected from the group consisting of indomethacin, piroxicam, ibuprofen, ketoprofen, naproxen, tenoxicam, etofenamate, mefenamine, flufenamic acid, felbinac, salicylic acid, acetylsalicylic acid, methyl salicylate, diethylamine salicylate and hydroxyethyl salicylate.

11. The method of claim 1, wherein the analgesic is diclofenac.

12. The method of claim 1, wherein the analgesic is present in an amount ranging from 0.5% to 40% by weight.

13. The method of claim 1, wherein the neuromuscular pain is a closed neuromuscular pain or joint pain.

14. The method of claim 1, wherein the neuromuscular pain is associated with a muscle bruise, muscle strain, muscle tear, back pain, rheumatic muscle complaint, myofascial pain, or tendonitis.

15. The method of claim 1, wherein the neuromuscular pain is associated with an inflammatory joint irritation, joint distortion or joint compression.

16. The method of claim 1, wherein the neuromuscular pain is associated with a local nerve irritation, nerve injury, or neurodermatitis.

17. The method of claim 1, wherein the neuromuscular pain is associated with an open wound or wound pain.

18. The method of claim 1, wherein the neuromuscular pain is associated with pain after surgery or post-incisional pain.

19. The method of claim 1, wherein said subject is human.

20. The method of claim 1, wherein said subject is a nonhuman animal.
